(19) United States
(12) Patent Application Publication
(10) Pub. No.: US 2005/0013740 A1
(43) Pub. Date: Jan. 20, 2005

(54) Computer-controlled automated titration apparatus for optically determining incompatibility of petroleum oils

(76) Inventors: Thomas G. Mason, Los Angeles, CA (US); Joseph Paszek, Rahway, NJ (US); Eric B. Sirota, Flemington, NJ (US); Mohankumar R. Sowlay, Phillipsburg, NJ (US); Lynda L. Smith, Somerset, NJ (US); James C. Sung, Flemington, NJ (US)

Correspondence Address: ExxonMobil Research and Engineering Company, P.O. Box 900, Annandale, NJ, 08801-0900, US

(21) Appl. No.: 10/865,441

(22) Filed: Jun. 10, 2004

Related U.S. Application Data
(60) Provisional application No. 60/480,955, filed on Jun. 24, 2003.

(51) Int. Cl. G01N 33/00

(52) U.S. Cl. 422/68.1

(57) ABSTRACT

An automatic titration apparatus for optically determining incompatibility of petroleum oils. The apparatus includes reservoirs for oil and solvent, a sealed temperature-controlled mixing vessel, including a liquid mixer, pumps and transfer lines for dispensing oil and solvent from said reservoirs to said mixing vessel wherein oil and solvent are mixed to form a mixture, an aggregate detector, transfer lines for transferring said mixture to the aggregate detector, and a computer programmed to operate and monitor all elements of said apparatus.

This application claims the benefit of Provisional application 60/480,955 filed Jun. 24, 2003.

### BACKGROUND OF THE INVENTION

The present invention is an automatic titration apparatus, including software for titration control. In particular, the apparatus and software determines the incompatibility of petroleum oils. Asphaltene aggregation can lead to the fouling of refinery process equipment and complications in the transportation and storage of crude oils residua, blending, and blends with process product streams, e.g. cracked stocks. Therefore, a test procedure is used to determine if mixtures of petroleum oils are incompatible and lead to asphaltene aggregation. One such test requires the measuring of incompatibility parameters.

Currently, the test procedure for measuring the incompatibility parameters consists of two tests: a heptane dilution test and; a toluene equivalence test. Based on microscopic observations of asphaltene aggregates in mixtures of the petroleum oil with different volume ratios of n-heptane and toluene, the insolubility number, I_{N}, and solubility blending number, S_{BN}, are calculated (see U.S. Pat. Nos. 5,997,723 and 5,871,634). These measurements require intensive human action in order to perform the mixing, make the microscope test slide, and observe whether or not asphaltene aggregates are present. The measurement for single petroleum oils typically takes three to four hours using the microscopic method.

Therefore, it is important to have an objective, reproducible, and more sensitive method for measuring the incompatibility of crude oils in order to predict whether or not asphaltene aggregates would form if two or more petroleum oils were blended together. It would also be advantageous to have a fast automated method of measuring the incompatibility with an instrument that would save hours of labor and eliminate the subjectivity of the microscopic method of measuring the incompatibility parameters.

### SUMMARY OF THE INVENTION

The present invention includes an automated titration apparatus and integrated software for titration control, asphaltene aggregation endpoint detection logic, and instantaneous reporting for optically determining the incompatibility of petroleum oils, petroleum resids, and petroleum oil mixtures. This instrument solves the existing problem of rapidly providing reproducible and objectively accurate incompatibility parameters, I_{N} and S_{BN}, for petroleum oils using a totally integrated automated titration system. It is designed to replace the manual microscopy method for determining incompatibility parameters for all crude oils including self-incompatible crude oils (see U.S. Pat. Nos. 5,997,723 and 5,871,634). The present invention includes an integrated software package, written in LabVIEW®, that controls all of the hardware components automatically and also senses the endpoint automatically without user intervention to stop the titration. Moreover, the present invention offers least squares reduction of a user-definable number of titrations for more accurate determination of the incompatibility parameters and instantaneous reporting of these parameters on the computer screen. All the measurement results are automatically stored on the computer's hard drive in spreadsheet-compatible format and a logfile is generated so that all conditions during the measurement are recorded as they occur. The present invention also offers a mechanical coupled mixer. This permits us to test viscous oils. The present invention also has an automated cleaning cycle so that the user can simply load an oil for testing into a dispensing syringe, press the start button on the computer software, and walk away from the apparatus, and the apparatus will measure the incompatibility parameters for that oil and report them without the user's presence or the cumbersome manipulation of raw spreadsheet data. The present invention also includes a closed mixing cell and titration loop that prevents evaporation of the oil and/or titration solvents. The present invention also can take several, user specified, titration points at a time. Again, the present invention offers an automated cleaning cycle and more advanced endpoint detection and automated reporting both on the screen and on the hard drive and can be printed out.

The present apparatus includes several subsystems, which operate together at the direction of a computer. The subsystems include a mixing system including a mixing vessel for mixing the substrate (such as a petroleum oil) and the solvents, a delivery system for delivering the substrate and the solvents to the mixing vessel, a temperature control system for controlling the temperature of the mixing vessel, a detection system that receives the substrate and solvents from the mixing system, a recirculation system to move substrate and solvent between the mixing vessel and the detection system, a computer programmed to automatically operate and monitor all systems of the apparatus. In a preferred embodiment, the computer automatically directs the apparatus from delivery of the substrate to the endpoint of one or more titrations, including cleaning the apparatus between titrations. All of the systems of the apparatus are discussed in more detail below.

### BRIEF DESCRIPTION OF THE DRAWINGS

FIG. 1 shows a schematic diagram of the computer-controlled/optical transmission spectroscopy automated titration (OTAT) apparatus.

FIG. 2(a) shows a flowchart of the main computer program that controls the devices which comprise the automated titration apparatus, processes signals obtained from the optical detector to detect the absence or presence of asphaltene aggregates, and reports the results to the computer's video display and to the hard disk drive.

FIG. 2(b) shows a flowchart of the subroutine used by the main computer program to perform a single computer-controlled titration, to automatically detect the endpoint of the titration using the optical detector, to automatically stop the titration, and to automatically clean the vial, cell, and flow lines.

FIG. 3(a) shows a portion of the front panel of the OTAT software covering user and sample information and operation parameters.

FIG. 3(b) shows a portion of the front panel of the OTAT software showing the automated setup screen by which the operator inputs oil and solvent mixtures. This screen also displays the results and plot of I vs. H as they are calculated.

FIG. 3(c) shows a portion of the front panel of the OTAT software showing the titration curve for Souedie oil titrated using n-heptane. The wavelength for the optical transmission spectroscopy was set at 750 nm. The maximum in the OT curve indicates that the transmission is dropping due to the scattering of light by the asphaltene aggregates

FIG. 3(d) shows the control screen which allows the operation of the pumps and mixers and monitoring of status of all the individual components of the system exclusive of the automated mode.

FIG. 3(e) shows a plot of the data from the OTAT software showing the measured incompatibility phase boundaries, corresponding to asphaltene aggregation for Souedie oil, least squares fits to these data, and the incompatibility parameters, I_{N} and S_{BN}, determined from the fit, along with the statistical errors in these values from the fit.

### DETAILED DESCRIPTION OF THE PREFERRED EMBODIMENTS

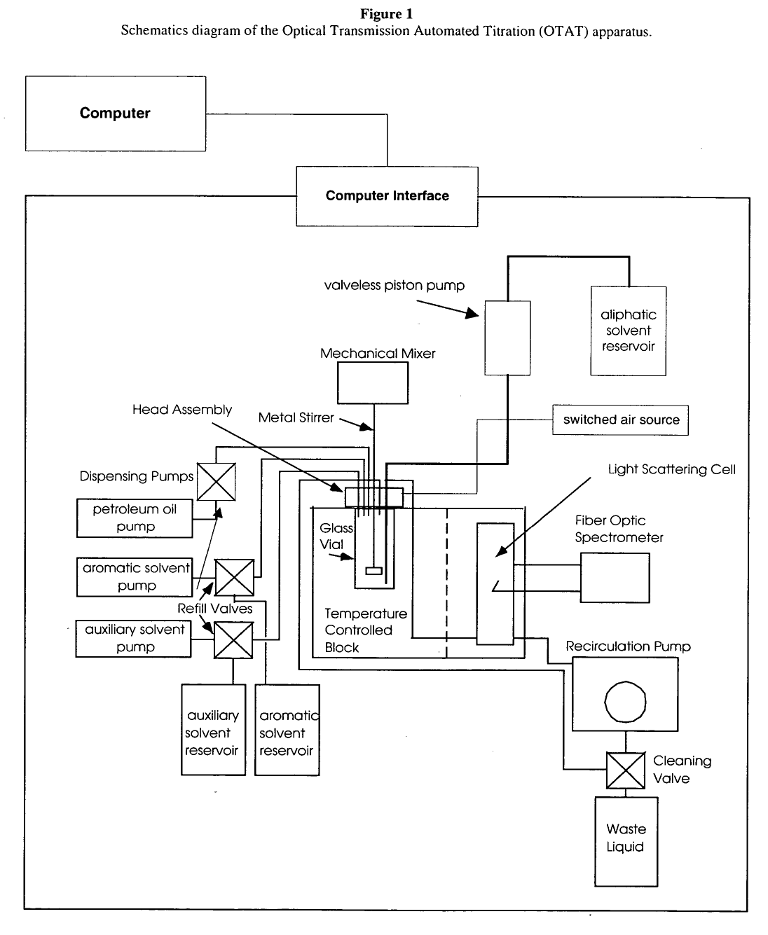

A schematic of the automated incompatibility apparatus of the present invention is given in FIG. 1. All components of the apparatus are completely computer controlled, and these components can be actuated and monitored through bi-directional electronic communication wires that link the computer and the components. Our implementation specifically relies upon the RS-232 communication protocol for all of the devices except the spectrometer, which uses its own proprietary communication protocol that is interpreted by a hardware card installed on the computer. The automated titration subsystem consists of bi-directional syringe pumps for extremely accurate dispensing of discrete volumes of oils or solvents, or continuous dispensing at a fixed rate, into a mixing vial. The titration pump for the system is a valveless piston pump, which, in a continuous stream, accurately delivers the titrant solvent into the titration vessel. Two position valves located at the head of the pumps permit the syringe pumps to either infuse into the mixing vial through Teflon® lines connected to the metal vial head assembly or to automatically refill by withdrawing liquid from reservoir jars connected to the valve. The vial is connected to the metal head assembly by the screw top of the vial and a Teflon® plate at the top of the adapter seals the vial so that evaporation is minimized. In the middle of the head assembly, there is a metal shaft with a propeller that is used to mix the contents of the vial; this metal shaft is connected to a flexible rotational cable, and this cable is connected to a mixing motor with a variable speed. The supply and return recirculation lines are kept in place by two short pieces of stainless steel tubing which keeps them clear of the mixing propeller. The recirculation subsystem consists of a variable speed peristaltic recirculation pump with a low dead volume and short length of hydrocarbon-resistant flexible tubing to facilitate the pumping action. The rest of the recirculation loop tubing is Teflon®. A cleaning valve is located in the recirculation loop, and, when it is switched on, permits the contents of the vial and the loop to be shunted into a covered liquid waste container. The heating subsystem consists of an insulated metal block with a cavity for receiving the vial and has internal metal heat exchanger lines containing three cartridge heaters. Two of the heaters provide temperature control for the titration cell and the third is for the spectrometer detector cell. The vial and metal head fits snugly against the top of the block containing the cell cavity. The cavity is typically filled with a liquid having good thermal conductivity (such as water) so that the contents of the vial reach temperature rapidly. The metal block also contains the two openings to allow viewing the contents of the titration cell. Thermocouples placed in the block allow for monitoring of the temperature of the titration and to assure that the temperature is maintained at the desired point. The entire heating block can be quickly dropped down away from the titration cell by releasing one pin on the system block. This arrangement allows for quick changing of cells during a manual run. The detection system consists of a fiber optic light transmission spectrometer (OT) arrangement. The liquid passes into a 100 micron thick optical cell that permits measurements by the optical spectrometer. The optical spectrometer subsystem consists of a tungsten white light source (filament temp≈3100 K), which is used to illuminate an optical fiber cable. This cable directs the light through the optical cell and a second fiber optic cable collects the transmitted light for wavelength separation by the spectrometer using a grating. The compact spectrometer (supplied by Ocean Optics Inc.) is connected to the computer through a proprietary card (also supplied by Ocean Optics). The typical oil volume required for a given titration is about 3-4 ml; since several titrations are needed to determine the incompatibility parameters of a petroleum oil, the total oil volume required to obtain the desired parameters is typically about 10 ml.

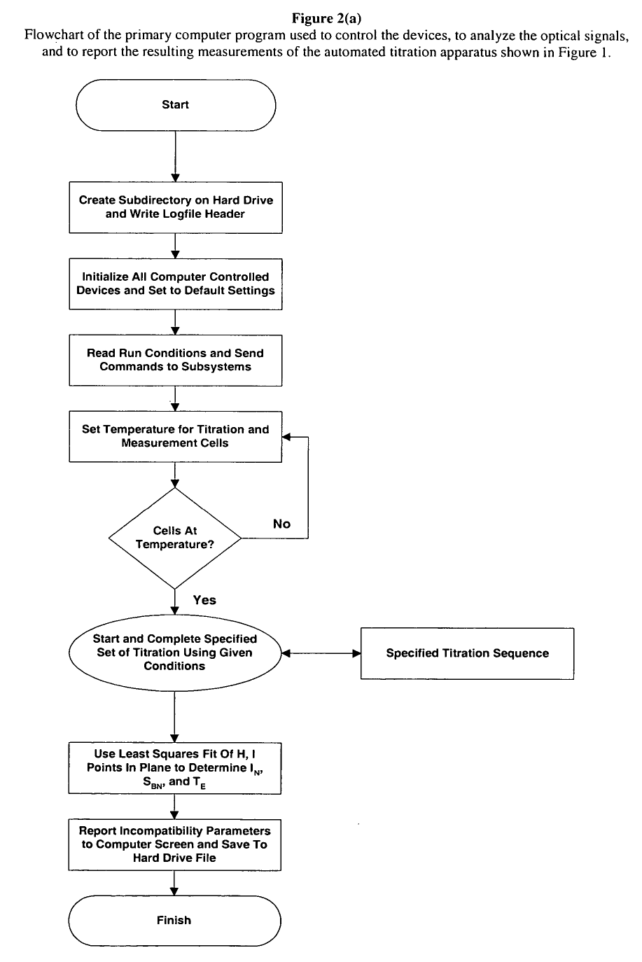

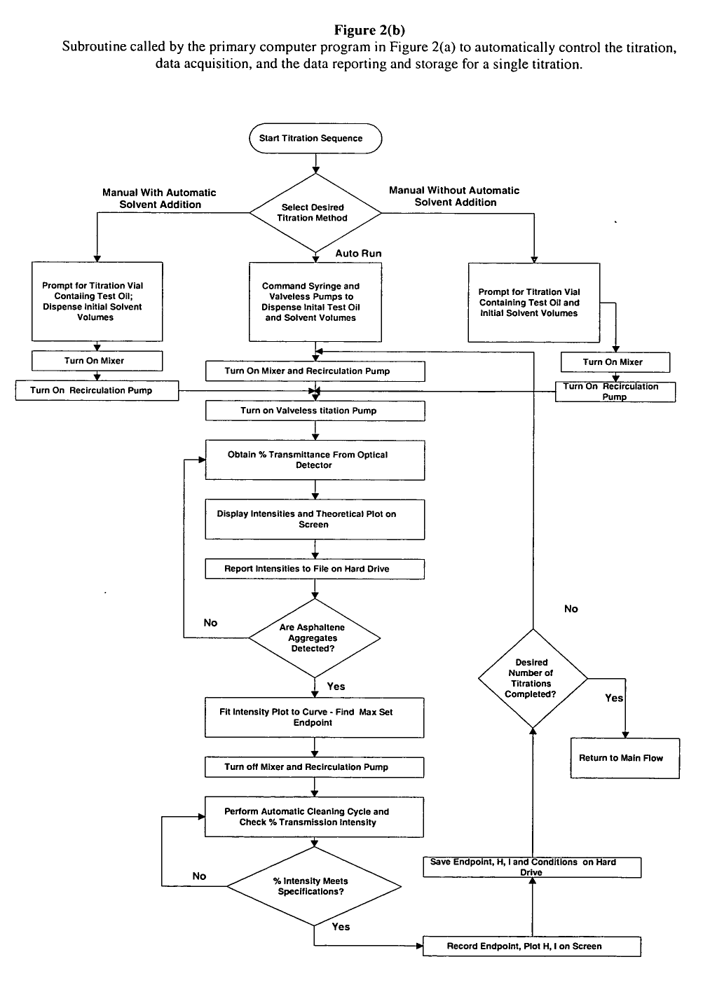

The general flowchart for the computer-controlled automated titration apparatus is shown in FIG. 2(a). This flowchart of the primary program provides the details about how to write a program in any software language to perform the computer controlled automated titration. The accompanying flowchart for the titration subroutine in FIG. 2(b) describes how a single titration can be controlled by a computer, and this subroutine is used by the primary program repeatedly in order to determine and report the incompatibility parameters for a test oil. Our particular implementation of this flowchart is in the graphical computer language known as LabVIEW®. It is emphasized that one of the most important aspects of the present invention is this integrated software package that has been developed for automatically controlling multiple titrations, monitoring the performance of the system, sensing the titration endpoint and automatically stopping each titration, cleaning the circulation loop, measuring the effectiveness of the cleaning of the circulation loop, calculating the incompatibility parameters using least squares fitting, instantaneously reporting the measurement results to the computer screen, documenting the history of each set of titrations in a logfile, and automatically saving the results in a proprietary format. This control program, currently written in LabVIEW® and called OTAT (optical transmission-automated titration), is the brains behind the instrument and enables the present invention to do what other commercial instruments cannot do at present. The user can control all of the dispense rates for the solvent and sample pumps, the mixer rate, the recirculation rate, the number of cleaning cycles, the titration aliquot dispense volume, and the initial volumes of oil and solvents loaded into the vial before the titration begins. The performance of the spectrometer is monitored and recorded during the start-up of the system and is used to confirm the operation of the spectrometer during titration operation. Similarly, the software provides for calibration of the titration pump to assure precise delivery of solvent. Moreover, the user can control the wavelength at which the titration curve is made. The present invention keeps track of the volume of liquid in the vial to prevent overfilling and will automatically stop a titration without recording an endpoint before the vial overflows. This is an important safety feature that allows the user to walk away from the instrument with confidence. The OT titration endpoint is reached and the program stops the OT titration when the transmission intensity passes through a maximum and decreases by a user-defined amount. The endpoint for the OT titration is specifically defined by the volume of aliphatic solvent (e.g. n-heptane) at which the OT transmission intensity is maximal, as determined by an automatically performed least-squares fit.

The software developed for the present invention offers three primary modes of operation for performing a titration, as shown in the subroutine in FIG. 2(b). The first, called “autofill”, gives the user the option of defining the number of independent titrations for determining the incompatibility parameters and entering the initial weights of test oil, aromatic solvent (e.g. toluene), and aliphatic solvent (e.g. n-heptane). When the user loads the oil syringe with the particular oil to be tested, clears air from the tubing lines, and presses the start button, the instrument automatically performs all the titrations in sequence and reports the results for I_{N} and S_{BN}, including the statistical errors from the fitting, without subsequent user intervention. This is an extremely convenient and time-efficient mode of operation. The second mode, called “manual with automatic pre-dilution”, permits the user to load the test oil into separate vials and to tell the program what the initial weights of the test oil are in each of the vials. The user then supplies the program with the weights of the initial dilution of the oil that are desired. The user does not add any solvents corresponding to the desired initial dilution into the vials since the program will do this automatically using the solvent pumps. When the user runs the program, the program prompts the user to screw each vial in turn into the head assembly. When a given vial is screwed in, the program commands the solvent pumps to dispense the desired solvent volumes before it starts the titration. This second mode saves the user time that would otherwise be required to manually add the initial dilution volumes of the solvents. The third mode, called “manual without automatic pre-dilution” or simply “manual”, permits the user to load the test oil and any desired initial dilution volumes of solvents into separate vials and to tell the program what the initial volumes of the test oil and solvents are in each of the vials. When the user runs the program, the program then prompts the user to screw each vial in turn into the metal head assembly and performs the titrations. This mode requires the presence of the user during the operation, and, although it is not as convenient, it permits the measurement of the incompatibility parameters of highly viscous oils, such as heavy crude oils and residua, which need some degree of pre-mixing with the solvent in order to facilitate the recirculation of the mixture.

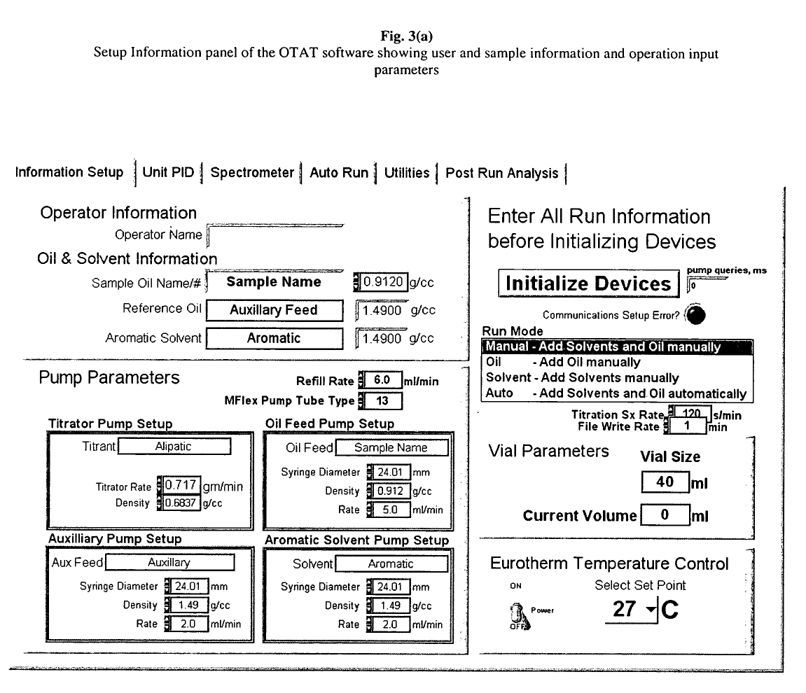

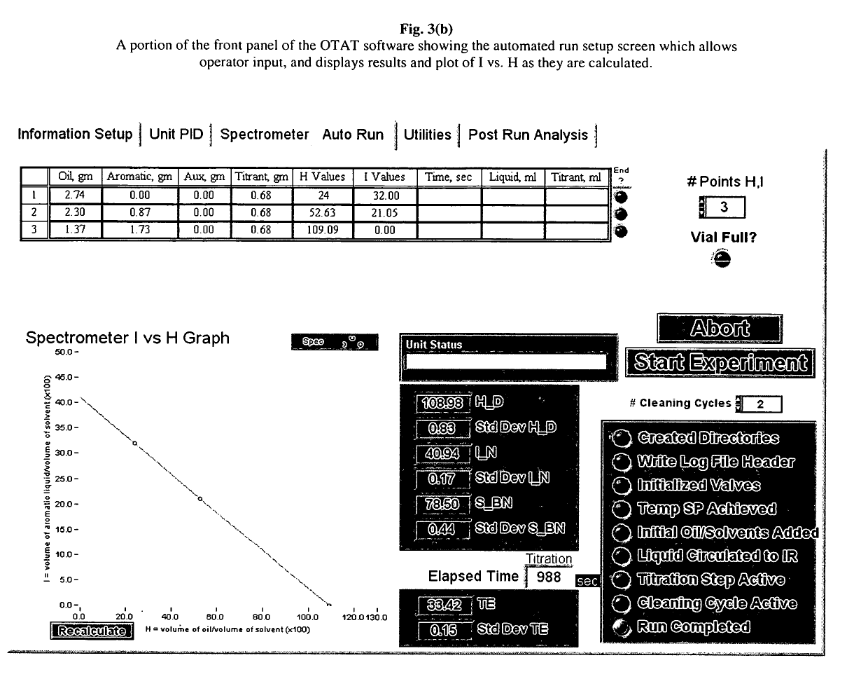

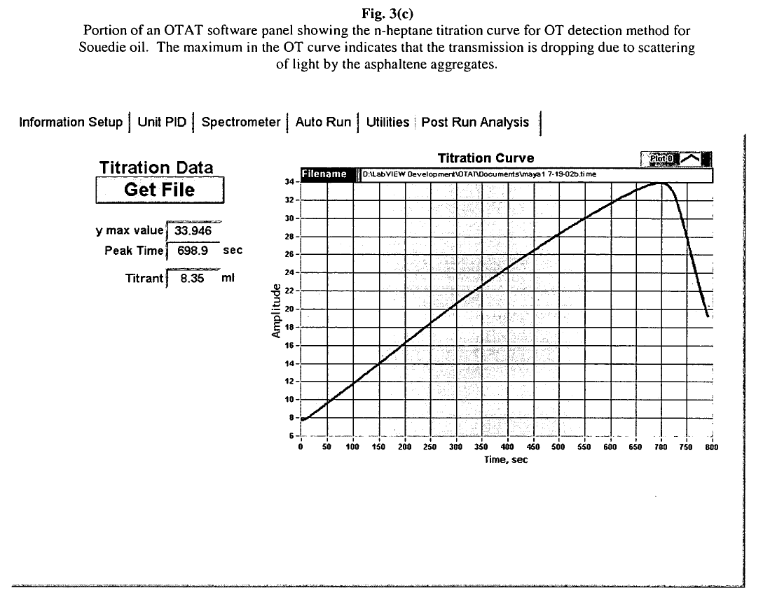

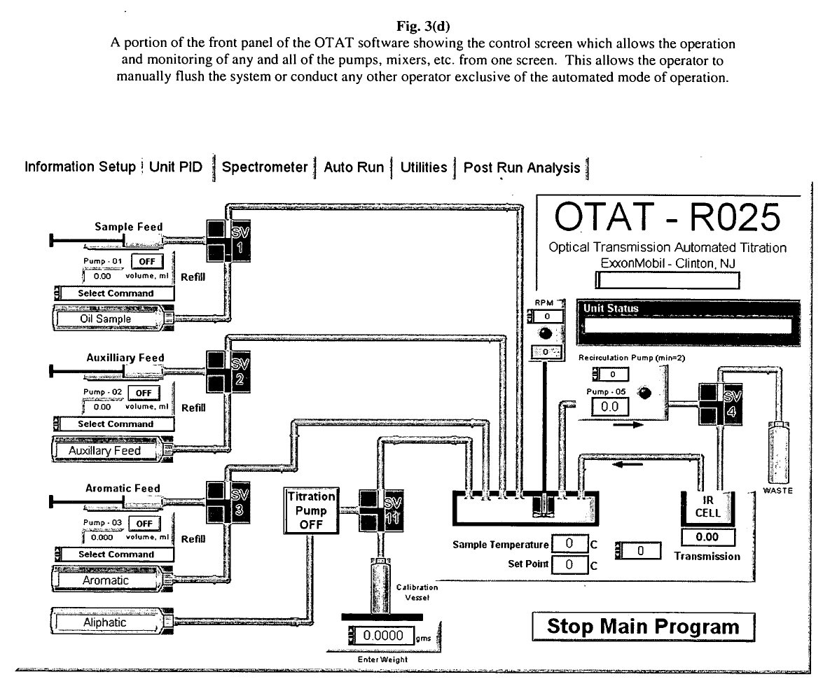

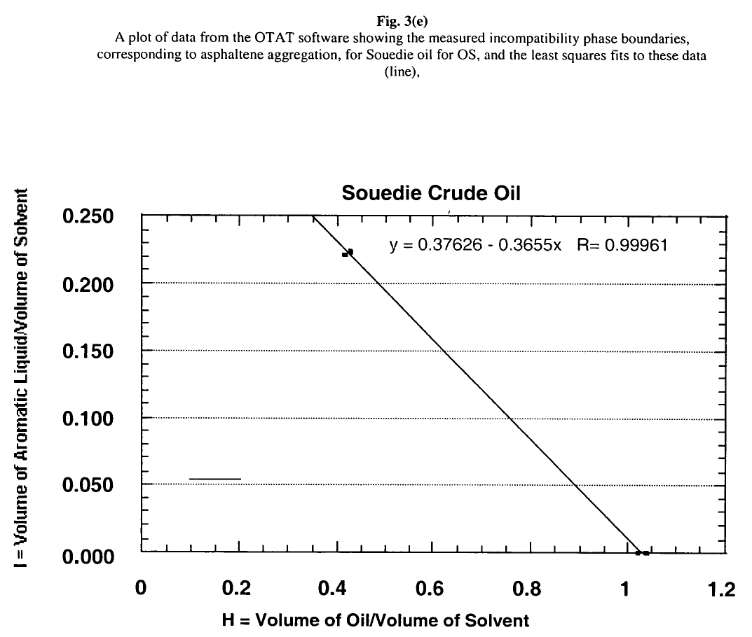

An example of the front panel of the software after a measurement of the incompatibility parameters for Souedie crude oil (in autofill mode with 4 titrations) is shown in FIGS. 3(a-d). FIG. 3(a) shows the user and sample information and the control settings; these are all recorded in the logfile. FIG. 3(b) shows an example of the OT measurements for the mixture after the last titration volume has been added. From each titration endpoint, the quantities H and I on the phase boundary can be calculated; H is defined as 100 times the volume of the oil divided by the total volume of the solvent, and I is defined as 100 times the volume of the aromatic liquid divided by the total volume of the solvent. Finally, the asphaltene incompatibility phase boundary for Souedie oil determined by all the titrations in the H-I plane is shown for OT detection data in FIG. 3(c). A least squares fit of these points is used to determine a straight line, from which the parameters I_{N} and S_{BN} are and displayed. If sufficient reproducibility of the points is not obtained, the program alerts the user.

A typical measurement of the incompatibility parameters for a crude oil requires about 1-2 hours with this instrument. We have successfully measured incompatibility parameters for light crude oils, heavy crude oils, asphaltene-free crude oils, self-incompatible oils, residua, and refinery products.

The advantages of this instrument over the prior technology include the following:
(a) Fast and user-independent method of measuring incompatibility parameters,
(b) Integrated computer control of all the components used for titration, mixing, heating, recirculation, calibration, and detection,
(c) Mechanical mixer to ensure mixture homogeneity, even for viscous oils,
(d) Computer-based logic for endpoint detection and process control (i.e. computer feedback in the titration process),
(e) Computer based logic for monitoring the performance of the spectrometer system and for calibration of the titration pump,
(f) Discrete oil and aromatic solvent aliquot dispensing with highly accurate syringe pumps (permits a wide range of titration rates),
(g) Continuous titrant solvent dispensing with a highly accurate valveless piston pump,
(h) Three modes of operation: autofill, manual with automatic pre-dilution, and manual without automatic pre-dilution,
(i) Availability of pre-defined protocol for measuring compatibility parameters for self-incompatible oils,
(j) User-defined number of titrations and initial titration volumes,
(k) Ability to add additional titration runs during the analysis sequence without having to stop the analysis.
(l) Small test oil volumes (3-4 ml per titration),
(m) Real-time on-screen reporting of raw measurement intensities, titration curves, and incompatibility phase boundary,
(n) Logfile documenting the entire history of the instrument during the determination of the incompatibility parameters,
(o) Incompatibility parameters are determined by least squares fit to the incompatibility phase boundary and reported,
(p) Automatic recording onto the hard drive of the raw measurement data, titration curves, and incompatibility phase boundary, and
(q) Automatic, computer-monitored, cleaning cycle and refill of solvent syringes
(r) A switched air source to minimize the amount of holdup solvent between titrations.

Although optical spectroscopy has been used to detect asphaltene aggregation (see Andersen, SI: “Flocculation Onset Titration of Petroleum Asphaltenes.” Energy & Fuels 1999 13:315-322), no existing commercial instrument offers these capabilities.

1. An automatic titration apparatus in which a substrate is titrated with one more solvent to an endpoint comprising:
(a) a mixing system including a mixing vessel for mixing said substrate and said solvents,
(b) a delivery system for delivering said substrate and said solvents to said mixing vessel,
(c) a temperature control system for controlling the temperature of said mixing vessel,
(d) a detection system that receives said substrate and solvents from said mixing system,
(e) a recirculation system to move substrate and solvent between said mixing vessel and said detection system,
(f) a computer programmed to automatically operate and monitor all systems from delivery of said substrate to said endpoint of one of more titrations of said apparatus.

2. The apparatus of claim 1 wherein delivery system includes pumps and transfer lines.

3. The apparatus of claim 2 wherein said pumps are syringe and valveless piston type pumps.

4. The apparatus of claim 3 wherein said delivery system further comprises reservoirs for said solvents so that said syringe pumps are automatically replenished with solvent when said pumps empty.

5. The apparatus of claim 1 where said detection system includes a spectroscopy cell.

6. The apparatus of claim 5 wherein said spectroscopy cell is temperature controlled by the temperature controlled system.

7. The apparatus of claim 1 wherein said recirculation system includes a cleaning valve and a waste liquid receptacle.

8. The apparatus of claim 1 wherein said computer automatically operates all systems from the delivery of said substrate to said endpoint of one or more successive titrations.

9. An optical automatic titration apparatus comprising:
(a) reservoirs for oil and solvent,
(b) a mixing vessel at a predefined temperature, including a liquid mixer,
(c) dispension system pumps and transfer lines for dispensing oil and solvent from said reservoirs to said mixing vessel wherein oil and solvent are mixed to form a mixture,
(d) an aggregate detector,
(e) recirculation system pump and transfer lines for transferring said mixture to and from the mixing vessel to the aggregate detector,
(f) a computer programmed to operate all elements of said apparatus.
(g) a switched air source to minimize the amount of solvent hold-up between titrations.

10. The apparatus of claim 1 wherein said pumps of 9(c) are syringe and valveless piston type pumps.

11. The apparatus of claim 1 wherein said pump of 9(e) is a peristaltic pump.

12. The apparatus of claim 10 wherein the recirculation system further comprises a cleaning valve and a waste liquid container.

13. The apparatus of claim 9 wherein said liquid mixer includes a mechanical mixer having a shaft extending into the mixing vessel with a propeller on its end within the mixing vessel.

14. The apparatus of claim 9 wherein said computer will automatically carry out a predetermined number of titrations.

15. The apparatus of claim 9 wherein said apparatus has a predetermined titration rate.

16. The apparatus of claim 12 wherein said computer has been programmed to dispose of said mixture, clean the system with aromatic solvent and refill the dispensing pumps.

17. The apparatus of claim 9 wherein said optical detector includes thin optical cell.

18. The apparatus of claim 17 wherein said optical cell is between 20 microns and 200 microns.

19. The apparatus of claim 9 wherein said computer determines incompatibility parameters of a petroleum oil.

20. The apparatus of claim 19 wherein said computer stores said incompatibility parameters for further reference.

21. The apparatus of claim 9 wherein said computer determines titration curves and asphaltene aggregate phase boundaries for real-time display.

22. The apparatus of claim 14 wherein said computer automatically detects the titration endpoint.

23. The apparatus of claim 9 wherein said computer allows three modes of operation, autofill, manual with automatic predilution and manual without automatic predilution.

24. The apparatus of claim 19 wherein said computer determines said incompatibility parameters using a least squares fit.

25. The apparatus of claim 9 wherein said computer provides real-time display of all graphical data.

26. The apparatus of claim 9 wherein said computer maintains a log file of all operations performed by said apparatus.

27. The apparatus of claim 9 where said computer is programmed to determine a toluene equivalence.

28. The apparatus of claim 9 wherein said optical detector is a spectroscopic detector.

29. The apparatus of claim 9 wherein said switched air source minimizes the amount of solvent hold up in the cell and recirculation system between titrations.
